(19) United States
(12) Patent Application Publication
(10) Pub. No.: US 2011/0178275 A1
(43) Pub. Date: Jul. 21, 2011

(54) INDUSTRIAL PLANT-BASED PRODUCTION OF ANIMAL-FREE RECOMBINANT PROTEINS IN DEFINED ENVIRONMENT

(76) Inventors: Bjorn Larus Orvar, Kopavogur (IS); Einar Mantyla, Rejkjavik (IS)

(21) Appl. No.: 13/001,669

(22) PCT Filed: Jun. 30, 2009
(86) PCT No.: PCT/IS2009/000004

§ 371 Date: Apr. 4, 2011

(30) Foreign Application Priority Data
Jun. 30, 2008 (IS) 8742

(51) Int. Cl. C07K 14/475; C07K 14/52; C07K 14/54; C07K 14/53; C07K 14/535; C07K 14/545; C07K 14/55; C07K 14/485; C07K 14/49; C07K 14/505

(52) U.S. Cl. 530/351; 530/399

(57) ABSTRACT

The present invention provides improved methods for animal-free protein production of high-value heterologous proteins produced in plants, plant derived tissue or plant cells. The invention reduces costs and increases the speed of manufacturing of active ingredients in transgenic plants. Furthermore, the invention improves the quality and safety of heterologous proteins produced in plants. The enhanced control of conditions in manufacturing of heterologous industrial and biopharmaceutical heterologous proteins obtained by the present invention with the combination of soil-less, hydroponic culture on conveyor belts with distinct nutrient zones in soilless greenhouses improves greatly the consistency of protein production in transgenic plants and conformity with quality control procedures applied for the manufacturing of active ingredients by the pharmaceutical industry, cosmetic industry, fine chemicals industry and veterinary industry.

### FIELD OF INVENTION

The present invention is within the fields of molecular farming, i.e. production of recombinant proteins in transgenic plants, such as in particular production of valuable protein biomolecules for medical or other use.

### BACKGROUND

Due to their complexity, heterologous proteins of high value are almost exclusively produced by living organisms that are able to fold the polypeptide backbone correctly and modify the folded heterologous polypeptide through post-translational modification to a varying degree, depending on the organism.

Protein based biopharmaceuticals show great promise in providing more specific and tissue specific, or cell specific drug treatments against serious diseases (for overview see “Recombinant Protein Drugs” Ed. P. Buckel 2001). Numerous examples in the prior art and applications have demonstrated the use of microorganisms such as bacteria, and animal cells, for the production of such biopharmaceuticals, of which insulin is a notable example. Many recent examples in the literature have demonstrated the utilization of transgenic plants or plant cell culture for expression and manufacturing of high-value heterologous polypeptides e.g. as biopharmaceuticals. Such plant-based manufacturing processes are referred to under the popular term “molecular farming.”

Production of valuable proteins can be made more economical with the use of plants as production organisms. The cultivation cost for plants used as host organisms for protein manufacturing can be considerably lower compared to most production systems based on bioreactors, such as prokaryotic production systems, animal cell cultivation and so forth. However, for all of the above production systems, purification of heterologous proteins remains a demanding and costly task.

However, plant expression systems have certain drawbacks. Introduction of foreign nucleic acid material can be difficult and limited to the available host species. Isolation and purification of heterologous proteins from plant matter can be cumbersome, depending on the protein being expressed and its affinity to non-soluble cellulosic plant matter. Hence, whereas upstream events in plant-based production look particularly promising, the downstream processing faces the same and In some respects more challenges than other expression systems used in the protein biotechnological production industry.

Outdoor field cultivation of transgenic plants for the purpose of molecular farming, i.e., production of high-values heterologous proteins, is the most economical way of obtaining the harvest as raw material for further processing and purification. However, field-conditions are poorly defined and very challenging with regard to confinement and quality control. The adoption of good manufacturing practices required by, for example, the pharmaceutical industry and regulatory authorities, is problematic. The plants may be exposed to extreme weather, plant diseases and insects affecting the physiology of the plants. In particular, it is difficult to control microorganisms and nutritional content of the soil that may or may not affect the plants health, adding to variables in cultivation.

Containment of the transgenic plants in a field can be very difficult with cross-pollinating plant species and requires vast buffer zones around the cultivation itself to capture pollen, increasing the area that is reserved for cultivation of such plants. Although self-pollinating plant species provide containment with regard to pollination, the implementation of Good Manufacturing Practices according to best pharmaceutical practices is challenging.

Greenhouses are traditionally used for the cultivation of fruits, flowers and vegetables and extensive development of cultivation technology has occurred through the years.

Molecular farming in closed environments such as greenhouses thus provides containment and the possibility of better control of conditions throughout the cultivation of protein-producing transgenic plants and production of high-value heterologous polypeptides, but at substantially higher cost. Greenhouse cultivation is however prone to high bioburden, where microorganisms, insects and fungi are present in soil used for the cultivation of plants.

It is of importance in the production of any substance to be used in pharmaceutical or scientific laboratory products that the production process be standardized and well defined. This puts stringent demands on the production of recombinant proteins.

Improved methods in molecular farming would be appreciated, where the entire process and conditions could be better controlled, in particular the chemical environment throughout the process. It is desired to obtain methods where stringent quality control can be more readily adopted, which may be required by regulatory authorities e.g. for the industrial production of biopharmaceutical polypeptides.

Existing safety concerns due to possible contamination with animal derived transmissible agents has created a need for recombinant protein products produced according to animal-free, and serum free principles, where the manufacturing process circumvents animal-derived components to the extent possible. Production of recombinant proteins in plants grown under animal-free conditions is ideally suited for this kind of animal-free, serum free production avoiding animal cells or serum as a source for the recombinant proteins or as a source of contaminants.

### SUMMARY AND OBJECTS OF THE INVENTION

The primary objective of the present invention is to provide an improved, contained and controllable method of large scale molecular farming, i.e. the industrial production of recombinant proteins in plants. This method makes the production of high-value heterologous proteins produced in plants, plant derived tissue or plant cells in greenhouses or closed confinements more efficient and suitable to quality control and good manufacturing practices. The method provides a process that is fully animal free consisting of animal-free cultivation of transgenic plants, expressing recombinant proteins.

The invention has the advantage of reducing the cost and improving both efficiency and quality of the cultivation of transgenic plants for the purpose of manufacturing of heterologous proteins expressed in plants.

The methods and system of the invention combine several features and technological advancements hitherto not used In the field of molecular farming. They offer much better control for ensuring that different individual plants are grown under substantially identical conditions in streamlined mass production.

The plants are cultivated in chemically defined media, that contains no animal derived components, soil or manure for the cultivation. The animal-free cultivation of the transgenic plants themselves provides a new level of safety to production of recombinant proteins and provides the biopharma industry with a solution to its safety concerns with regard to the contamination risk of recombinant products with transmissible agents.

An important step in the cultivation process is the use of hydroponic conveyor belts that facilitate large scale cultivation of transgenic plants, enabling the movement of the plants from seedling stage at one end of the conveyor belt to the other end of conveyor belt where the plants are ready for harvesting.

With the use of soil-free hydroponic technology coupled with conveyor belt green houses, the production of transgenic plants on a large scale remains under controlled conditions. The present invention provides a novel process and system that are more readily adapted to pharmaceutical and biotechnological industrial production standards, yet allowing industrial scale production of valuable biological compounds.

Hydroponic greenhouse technology has been used for foodstuff production, seeking advantages of higher efficiency, i.e. fast growth and efficient use of resources, in particular, water and nutrients. Hydroponics used in molecular farming provides additional advantages, mainly by providing a clean and much more controllable environment, beneficial for standardizing the production processes and keeping the production facilities as clean as possible, minimizing bioburden from insects, microbes, fungi and other. external pollutants.

Plant-based production of proteins shows great promise for large scale manufacturing of proteins in an economic manner, as has been shown by examples in literature (for overview see Hammond 1999). The cultivation costs involved in molecular farming with plants are considerably lower than with traditional bioreactor-based methods. The use of plants as an expression system for production of valuable heterologous proteins, such as mammalian, e.g. human proteins, offers several unique advantages, including high production yields at competitive low cost, reduced health risks from pathogen contamination, and correct modification and assembly of foreign proteins. An additional advantage of a plant production system is that proteins, in the case of oral immune tolerance induction, may be used directly after separation from the bulk plant material without extensive purification, resulting in further cost reductions, as compared to animal or bacteria-based expression systems.

A useful feature of the present invention provides for the control of the cultivation through the precise application of nutrients, that can be varied according to the developmental stage of the transgenic plants. The invention makes use of conveyor belts, conveying, in a preferred embodiment plants at a suitably slow speed through different zones of differential irrigation. In the different zones, different composition and/or concentration of nutrient solution can be provided, adjusted according to the developmental stage and needs of the plants in each zone. Further, different irrigation schemes can be applied in the different zones, meaning the irrigation can be provided for different periods of time and/or at different time intervals. Such different schemes are illustrated in the Examples provided.

In some embodiments, the plants are conveyed through at least three zones, e.g. three, four or five zones. The speed of the conveyor belt is adjusted suitably based on the growth rate of the plant species and variety being grown, so that a plant will move along the entire conveyor from seedling state until it is ready and suitable to be harvested. Typically, the plants will travel the entire length of the conveyor band in a number of weeks or days, for example, for about 4-20 weeks, such about 6 weeks, 8 weeks, 10 weeks, 12 weeks, 14 weeks, 15 weeks, 16 weeks or about 18 weeks, depending on the plants being grown.

Different plant species and/or variants within the same species can be grown simultaneously, provided they reach efficient harvest size at a common timepoint from being placed on the conveyor, in particular if they have similar nutritional needs, such that one common Irrigation system can be used.

Another important feature of the invention which substantially increases conformity in molecular farming and facilitates quality control and simplifies process control, is the exclusion of soil from the cultivation, with the use of inert inorganic material as support for the plants and chemically defined plant nutrition media.

The methods of the invention further provides in some embodiments, further control of conditions, such as but not limited to control of UV light, by use of automatically controlled sun shade panels and/or electric UV lights.

The transgenic plants used in the methods of the invention can be any plant amenable for introduction and expression of foreign DNA, provided the plants can be grown hydroponically. Consequently, useful plants include both dicotyledonous plants and monocotyledonous plants and may be common agricultural plants known to be genetically modifiable, including tobacco, rape seed, soy bean, lettuce, alfalfa, barley, maize, wheat, oat and rice.

The methods are preferably used with plants which can express the heterologous protein within its seeds, such as common cereal plants, including barley, wheat, oat, rice and the like. As demonstrated in the examples herein, transgenic seeds containing the heterologous protein of interest serve as an excellent storage media, where the heterologous protein remains fully stable and active for an extensive period even if stored at room temperature, adding much flexibility to the production process.

In preferred embodiments of the present invention, the transgenic plant is selected from a self-pollinating species such as but not limited to barley, which minimizes the requirement for preventive measures due to possible cross-pollination, such as buffer zones of non-transgenic plants, physical separations in form of walls or curtains or bags placed on the plants or the like, in order to restrict pollen flow.

The invention has the advantage of increasing productivity and numbers of harvests. This is exemplified by increase in productivity as well as the number of harvests of barley grain per year, which results in up to 5 harvests per year of transgenic grain containing a heterologous polypeptide. Therefore, the present invention greatly enhances quantities produced of the heterologous polypeptide in a given time period and plant mass, and maximizes the efficiency of production area and facility. This feature streamlines and improves the economy of molecular farming.

The transgenic plant can be obtained from tissue culture or from propagated material such as seeds; and are generally planted in the inert matrix, which is wetted with water or nutritional solution.

In certain embodiments of the invention, the production method uses only renewable energy sources, such as geothermal heating of the production facility, in addition to natural light lighting energy for photosynthesis and all electric power used for the production is powered by electricity generated with hydropower or geothermal power. This adds to the sustainability and energy efficiency of the production method described by the invention, and makes the whole method of production uniquely sustainable.

The matrix used for molecular farming according to the invention provides support and firmness for the roots while being porous, enabling the roots of the plant to reach nutrition. Thereby, more economical and safer growth conditions are enabled for plant derived heterologous proteins. Examples of useful matrix material include but is not limited to volcanic pumice, light expanded clay aggregate (LECA), rockwool, glasswool, perlite, coir, vermiculite, sterilized sand, washed gravel, and polystyrene peanuts. Suitable matrix material can be readily selected by the skilled person, depending on the plant being grown, as well as practical considerations (local availability, costs, etc.)

It is a further advantage of the present invention that the transgenic plant material obtained by the method of the invention, and harvested to be processed further, is of superior quality for the purpose of extracting or purifying the respective heterologous polypeptide. This is evident by comparing the endotoxin content of two batches of heterologous proteins, one produced by the method of this invention and the other with conventional expression by bacteria. The results demonstrate superior quality of the heterologous polypeptide product produced according to the present method and less risk of pyrogenic inflammatory response upon contact with animal or human cells or tissues.

It is also an advantage of this invention to provide transgenic plant material that is superior as a raw material for production of heterologous proteins, as compared to transgenic plants produced in conventional manner, in the field or in greenhouses, as a result of the improved control of the process. Furthermore, the plants are grown in chemically defined, animal-free media under extensive automation. Such further control of conditions provide, not only less bioburden and undesired contamination, but also more homogeneous protein product, less batch-to-batch variations, e.g. with regard to concentration of the protein in plant tissue, homogeneity of post-translational modification, to name a few advantages.

In certain embodiments of the invention, it is an advantage of this invention to direct the expression of the gene encoding the heterologous recombinant protein to seeds. This provides for exceptional flexibility in production of recombinant proteins, as the seeds containing the product scan be stored for years before processing and purifying the protein without affecting the quality of the heterologous protein. Thus, the product can be stored and stock-piled in a stable form, in sterile environment within the seeds, and a decision on further processing and purification can be taken based on demand for the particular heterologous protein. This feature, intrinsic to the technology of the present invention, results in more economical, cost effective and competitive production of valuable heterologous recombinant proteins.

In preferred embodiments, the transgenic plant or plant cell comprises any nucleic acid sequence encoding for a heterologous polypeptide such as, but not limited to growth factors, cytokines, enzymes, monoclonal antibodies, that is expressed in the plant or plant cell and the polypeptide is produced by the plants under the conditions described by the present invention. Any proteins, which can be expressed in plants are within the scope of the invention. In this regard, plants provide advantages over other expression systems such as prokaryotic systems due to the possible post-translational modification of proteins in plants. Hence, the method of the present invention is particularly suitable for proteins that are not readily expressed fully active in prokaryotic systems, including for example valuable mammalian proteins such as, growth factors, which may be derived from humans or other animals, including mammals, such as rodent (e.g. mouse derived), pig, cow, goat, to name a few. Growth factors may include, but are not limited to, the following: Transforming Growth Factors-b (or beta) (TGFs-b or TGFs-beta), Transforming Growth Factor-a (or alpha) (TGF-a or TGF alpha), TNF alpha, Epidermal Growth Factor (EGF), BMP-4, Platelet-Derived Growth Factor (PDGF), KGF, Fibroblast Growth Factors a and (aFGF and bFGF), Vascular Epithelial Growth Factor (VEGF) Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-7 (IL-7), Interleukin-6 (IL-6), Interleukin-8 (IL-8), Interleukin-10 (IL-10), Interleukin-18 (IL-18), Interleukin-20 (IL-20), Tumor Necrosis Factor-a (TNF-a), Tumor Necrosis Factor-b (TNF-b), Interferon-g (INF-g),Granulocyte Colony Stimulating Factor (G-CSFs), Granulocyte Macrophage Colony Stimulating Factor (GM-CSF), Macrophage Colony stimulating factor (M-CSF), Nerve Growth Factor (NGF), Keratinocyte Growth Factor (KGF), Bone morphogenesis Protein (BMP-4), and Thymosin beta 4, and all isoforms thereof.

In a highly preferred embodiment of the invention, a heterologous polypeptide of interest being produced in the transgenic plants with the technology of the present invention, contains an affinity tag at either N-terminal or C-terminal of the polypeptide, or at both ends. Such a tag may include repetitive HQ sequence, poly Histidine-tail, GST, CBM or any other useful affinity tag that simplifies purification of the heterologous peptide.

The present invention successfully addresses the short-comings of expensive, costly and potentially less safe methods for the production of valuable heterologous proteins for non-food, industrial purposes, allowing for the contained cultivation of transgenic plants under controlled conditions for the production of valuable heterologous proteins at small, medium and large scale, for purposes such as, but not limited to, chemical industry, cell media industry, cosmetic industry, biotechnology industry and the production of protein-based pharmaceuticals.

In particular, it provides a novel process of producing heterologous proteins from biomass such as plant-derived material, with fewer processing steps involved, taking advantage of safer and more economical and sustainable production principles.

The present invention importantly is a process that facilitates quality control and therefore amenable for use within the pharmaceutical industry, the cosmetic industry and the fine chemicals industry. Accordingly, proteins produced by the present invention can be used in pharmaceutical products, cell media compositions, cosmetic products, as ingredient in laboratory products, and the like, as well as for producing industrial enzymes, and more.

Methods for introducing and expressing foreign genes in plants are well known in the art. A plant that can be genetically transformed is a plant into which heterologous DNA sequence, including DNA sequence for a coding region, can be introduced, expressed, stably maintained, and transmitted to subsequent generations of progeny. Genetic manipulation and transformation methods have been used to produce barley plants that are using herbicide resistance including, for instance, bialaphos or basta, or antibiotic resistance, such as hygromycin resistance, as a selectable marker.

Suitable cultivars are selected and a suitable method for introduction of foreign gene selected. The term “transformation” or “genetic transformation” refers to the transfer of a nucleic acid molecule into the genome of a host organism, resulting in genetically stable inheritance. Host organisms containing the transformed nucleic acid fragments are referred to as “transgenic” organisms. A “transgenic plant host cell” of the invention contains at least one foreign, preferably two foreign nucleic acid molecule(s) stably integrated in the genome. Examples of methods of plant transformation include Agrobacterium-mediated transformation (De Blaere et al. 1987) and particle-bombardment or “gene gun” transformation technology (Klein et al. (1987); U.S. Pat. No. 4,945,050).

WO 2006/016381 describes a particular useful Barley cultivar amenable for transformation and describes in detail suitable transformation methods.

WO 2005/021762 discloses methods for modifying proteins by making chimeric proteins that are readily purified on a large scale.

### BRIEF DESCRIPTION OF DRAWINGS

FIG. 1 demonstrates a purified heterologous polypeptide Granulocyte colony stimulating factor (G-CSF) produced by the present method of production.

FIG. 2 demonstrates a difference in endotoxin levels of a growth factor produced with the method described by the present invention and a bacterially produced growth factor.

FIG. 3 shows activity of human VEGF protein purified from transgenic barley seeds after (a) 16 months, and (b) 3 months, of storage at room temperature of the seeds. Graph (c) shows for comparison activity of bacterially produced VEGF, purified directly from bacterial culture.

### DETAILED DESCRIPTION OF THE INVENTION

Unless defined otherwise, all technical and scientific terms used herein have the same meaning as is commonly understood and used by one of skill in the art to which this invention belongs.

The term “polypeptide” used herein refers to any polymer of amino acids, being monomeric or multimeric, and does not refer to a specific length of a polymer of amino acids. Thus, for example, the terms peptide, oligopeptide, protein, and enzyme are included within the definition of polypeptide. This term also includes polypeptides with post-expression modifications such as for example, glycosylations, acetylations, phosphorylations and the like.

The term “heterologous polypeptide of interest” or “polypeptide of interest” used herein refers to any polypeptide intended for expression in plant-cells or plant tissue using the methods or compositions of the present invention. As non-limiting examples, pharmacological polypeptides (e.g., for medical uses, for cell- and tissue culture) or industrial polypeptides (e.g. enzymes, growth factors) can be produced according to the present invention.

The term “expression” and “production” refer to the biosynthesis of a gene product, including the transcription and translation of said gene product.

“Molecular farming” refers to the operation of using plants of any kind in open fields or in a closed facility to express and produce heterologous proteins in their tissue.

“Animal-free” refers to avoidance of components of animal origin in the process described by the invention and prevention of such components to come in contact with the heterologous recombinant protein product, or the plants used for production of the protein. Animal-free also encompasses the origin of the DNA used for transforming the plants: The gene is not isolated from animal or human source but is chemically synthesized according to available sequence information.

The term “controlled environment” is used in this context to describe environmental conditions for cultivating plants where chemical and physical conditions can be controlled, including irrigation and nutrition and preferably also temperature, humidity and carbon dioxide content, which is soil-less.

The term “GMP” (good manufacturing practice) is well known in the art and dictates the manner in which biopharmaceuticals and other drugs and medical devices are produced. GMP requirements include standard operating procedures, sterile conditions, validation of materials and equipment and trained personnel.

The term “transgenic” as used herein refers to any cell, cell line, plant tissue, organ or organism into which a non-native nucleic acid sequence has been introduced, and thereby altering its genotype; the term can also refer progeny thereof in which the non-native nucleic acid is present.

Typically, the non-native nucleic acid sequence was introduced into the genotype by a process of genetic engineering, or was introduced into the genotype of a parent cell or plant by such a process and is subsequently transferred to later generations by sexual crosses or asexual propagation.

The term “isolated” is used herein in a broad sense referring generally to material that is separated partially or fully from its source of origin; accordingly, isolation of a heterogeneous protein from a plant in which it is expressed can refer to partial or incomplete purification, e.g. harvesting and milling of seeds from plants which express heterologous protein in their seeds, harvesting of fruit containing heterologous protein, etc.

The term “transformation” or “transformed” refers to the introduction of a nucleic acid sequence into the DNA genome of a host organism, irrespective of the techniques used for the introduction of the nucleic acid fragment into the host cell.

The invention provides in a first aspect a process for producing a heterologous protein in a transgenic plant in a controlled environment, wherein the process comprises at least the following steps:
cultivating hydroponically in a greenhouse transgenic plants in an inert soil-free matrix, which plants express in at least part of their tissue said heterologous protein,
conveying on a conveyor belt gutters containing the transgenic plants in said inert matrix, through different zones of irrigation, and wherein newly sowed or planted plants are placed at the front end of said conveyor belt, and harvesting occurs at the back end of the conveyor belt,
harvesting said transgenic plants,
separating from said harvested plants said heterologous protein.

Preferably, the controlled environment is animal-free, as further defined herein.

Preferably, however, the contained process encompasses all steps starting from transgenic seeds until and including harvesting of the plants and preferably at least some initial steps of separation of the heterologous protein from the bulk plant material. Thus, the process may encompass the steps of
sterilising transgenic seeds of said transgenic plant,
sowing said seeds in said inert matrix,
when the seeds have germinated, the seedling are placed on the front end of the conveyor and conveyed through irrigation zones as described above.

The transgenic seeds may be “primed” (hydroprimed) before sterilization, such as by soaking the seeds in water, e.g. for 24 hours. Seeds can be sterilized by methods known in the art, suitably soaking in ethanol solution as is explained in the accompanying example, and dried.

After sterilization and drying the primed seeds, the seeds are suitably sown in the inert matrix and the seeds allowed to germinate. The germination can preferably take place in a germination chamber with high humidity. In case of barley seeds, this may take up to six days. In certain embodiments, the germination is allowed to continue outside the germination chamber and the pots with germinating seeds watered to prevent drying.

The conveying belt and nutrient zone irrigation system offers a high-throughput system where plants with different transgenes expressing different heterologous proteins can be grown simultaneously in the same system.

Monocotyledonous and dicotyledonous plants that can be genetically manipulated can be used in the present invention. Preferably the plant is a monocotyledonous, more preferably barley, and most preferably the barley Hordeum vulgaris. A plant that can be genetically transformed is a plant into which non-native DNA sequence, including DNA sequence for a coding region, can be introduced, expressed, stably maintained, and transmitted to subsequent generations of progeny. Genetic manipulation and transformation methods have been used to produce barley plants that are using herbicides including, for instance, bialaphos or basta, or antibiotic, such as hygromycin, as selectable markers.

Preferred embodiments of the invention make use of transgenic plants which express the heterologous protein in their seeds. This greatly simplifies the handling of the protein after harvesting of the plants, as the protein can be stored in the seeds for quite extensive periods of time until when it is suitable to make use of the protein, e.g. selling or introducing into another product. The examples herein demonstrate that heterologous protein, (as illustrated with human Vascular endothelial growth factor) remain active even after 16 month of room temperature storage of the seeds. This means that heterologous proteins can be stockpiled, using the transgenic harvested seeds as a convenient storage medium.

The step of separating from the harvested plants said heterologous protein broadly encompasses partial separation of plant material with little or no heterologous protein from plant material that contains said protein. Thus, separation includes separation of leaf material or other material which does not contain heterologous protein, or as in the accompanying examples, harvesting and threshing plants to collect seeds which contain heterologous protein. The separation step may in other embodiments also encompass further processing and or separation, such as but not limited to milling, and/or further protein purification steps such as chromatography steps.

Hydroponic cultivation refers to methods of growing plants using mineral nutrient solutions instead of soil. In this manner terrestrial plants may be grown with their roots in the mineral nutrient solution only or in an inert medium. Hydroponic technology has been used in greenhouse farming of vegetables and fruits. Hydroponic technology enables much more efficient use of water and nutrients and provides for a cleaner environment, reducing need for plant protection agents (insecticides, etc.). Hydroponics is frequently used in biology research.

Preferred embodiments of the invention use chemically defined, notably animal-free nutrient solutions instead of nutrient solutions containing salts, minerals or other components from animal sources or poorly defined sources. The method of growing the transgenic plants in an animal-free nutrient solution under controlled conditions according to the present invention presents a unprecedented level of safety and a greatly improved level of quality to those sectors of industry and academia requiring recombinant proteins as intermediary or final components or parts of compositions or processes and striving for animal-free manufacturing of products or applications such as, but not limited to, pharmaceuticals, biopharmaceuticals, cosmetics, cell culture media, stem-cells, for applications within regenerative medicine, cell culture media and fine chemicals and the like.

In another aspect, the invention provides a system for producing heterologous protein from transgenic plants in a greenhouse, comprising transgenic plants as described above, that express in at least a portion of their tissue, and preferably in their seeds, said heterologous protein, which is suitably a protein selected from any of the above mentioned, and which system further comprises a conveyor belt with gutters for holding said plants in soil-free inert matrix as described above, and an irrigation system which divides the conveyor belt area into zones as described above. The system preferably also contains a germination chamber and means for threshing harvested plants, all within confined clean environment, for production desired protein products in well defined settings.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLE

### Example 1

### Contained Soil-Less Molecular Farming in Hydroponic (Herein Referred to as the Heklagro™ Technique)

Cultivation with the Heklagro™ technique starts with the priming (hydropriming) of the transgenic seeds. This was done by soaking the seeds in water for 24 hours. Following the priming the transgenic seeds were sterilized (70% ethanol for 1 minute, 1.5% sodium hypochlorite (200 ml+2 drops Tween20) for 10 minutes, rinse 5× with sterile water) and then dried over night in laminar flow cupboard.

Following priming and sterilization the transgenic seeds were brought to the greenhouse where they were sown into pots filled with wet volcanic pumice. After sowing the pots were stored in a germination chamber (19.5-24° C., 70-90% humidity) until the first leaves start to emerge or up to 6 days. Subsequently, the pots were removed from the germination chamber and placed on roller benches where the germination continues. The pots were watered every day so that the pumice does not dry.

Finally the pots were placed into their positions on the conveyor belt, which was divided into nutrient zones. For the first 5 weeks the plants go through zone 1 where they were watered with nutrient solution every 120 minutes for 5 minutes. The animal-free nutrient solution contains fertilizer, and suitable nitrate source. Full strength solution contains: N, P, K, Mg, S, Ca as well as micronutrient Fe. In zone 2 the plants only get half strength solution for 5 minutes every hour and in zone 3, every 4 hours. The plants go through zone 2 in 6 weeks whereas zone 3 lasts four weeks. When the transgenic plants reach the end of the conveyor belt, they are fully ripe and ready for harvest.

Granulocyte colony stimulating factor (G-CSF) is an example of a heterologous polypeptide produced according to contained soil-less molecular farming in hydroponic culture on conveyor belts. The figure illustrates that G-CSF was produced and accumulated as the production vehicle, in this case a barley plant, moves along the conveyor belt.

Harvested transgenic seeds were threshed and the seeds were dried at ambient temperature under forced airflow for 72 hrs to standardize the water content of the harvested transgenic seeds. After drying the transgenic seeds were split into samples that were for long term storage and seed banking, and to batches for processing. Long term storage and transgenic seed banking samples were placed in aluminum coated vacuum bags that provide efficient protection from light and the bags were sealed under vacuum, labeled with barcodes and stored at −20° C. for long term storage. The batches of grains for processing were surface sterilised with 80% ethanol, washed five times with distilled water and dried overnight before milling and further processing and purifying of the heterologous protein.

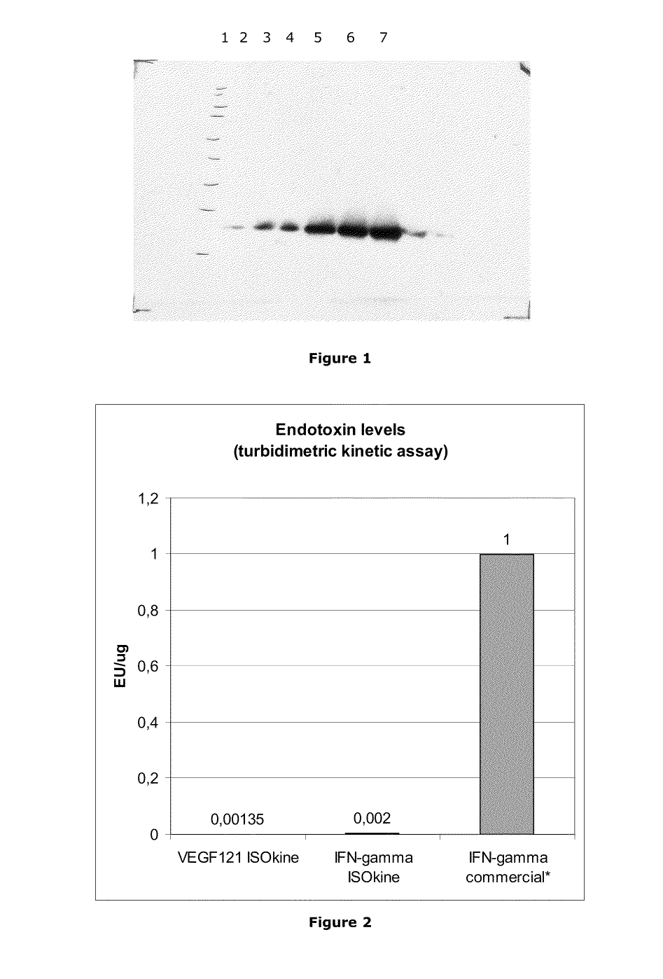

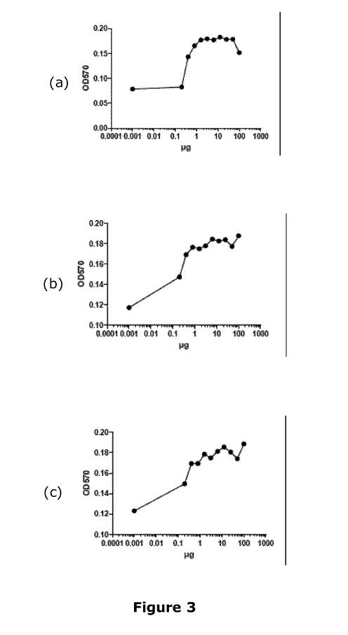

FIG. 1 demonstrates the production of heterologous polypeptide (G-CSF) in barley grains according to the invention's contained soil-less molecular farming method in hydroponic culture on conveyor belts. The production of the heterologous protein in G-CSF producing transgenic barley line was monitored at different points along the conveyor belt, reflecting different developmental stages of the transgenic barley. For monitoring, western blotting was used with total extract of barley grains and partially purified using G-CSF specific antibody (Autogen Bioclear, UK). Lane numbering: Lane 1 Size marker, lane 2 extract harvesting timepoint 1 , lane 3 extract harvesting timepoint 2, lane 4 extract harvesting timepoint3, lane 5 timepoint 1 partially purified, lane 6 timepoint 2 partially purified, lane 7 timepoint 3 partially purified.

### Example 2

### Heterologous Protein Safely Stored in Barley Seeds

Mature transgenic barley seeds containing heterologous human Vascular endothelial growth factor (VEGF) were harvested, threshed and dried. The seeds were stored at room temperature in weaved nylon bags for 16 months before milling followed by subsequent extraction to aqueous phase. The extract was spun down to clarify the extract, and the clarified extract was exposed to series of chromatography matrices during purification. The final purified VEGF product was aliquotted to vials and freeze-dried.

The freeze-dried VEGF was tested for activity by performing MTT Cell Proliferation Assay where serial dilutions of reconstituted recombinant plant-derived VEGF are applied onto primary HUVEC cells for cell proliferation bioassay. The bioassay resulted in atypical sigmoidal curve that enabled calculation of 50% effective concentration (EC50). The EC50 was determined to be EC50=0.29 ng/mL for Human VEGF, which is comparable to VEGF purified from barley seeds three months post harvesting EC 50=0.19 ng/ml. This can be compared to bacterially produced recombinant human VEGF that exhibited activity of EC 50=0.18 ng/ml.

This example shows that recombinant protein produced according to the present invention retains full activity after extensive storage in the form of seeds, illustrated here with over 16 months storage time at room temperature.

Although only preferred embodiments of the invention are specifically illustrated, numerous modifications and variations in the invention as described in the above examples are expected to occur to those skilled in the art, without departing from the spirit and intended scope of the invention.

It will of course be realised that while the foregoing has been given by way of illustrative example of this invention, all such and other modifications and variations thereto as would be apparent to persons skilled in the art are deemed to fall within the broad scope and ambit of this invention as is herein set forth.

Throughout the description and claims this specification the word “comprise” and variations of that word such as “comprises” and “comprising”, are not intended to exclude other additives, components, integers or steps.

### REFERENCES

Boraston et al. (2001) Biochemistry 40, pp. 6240-6247.

Contributors (2001) in “Recombinant Protein Drugs” Ed. P. Buckel—from series—Milestones in Drug Therapy, Birkhauser Verlag, Basel 2001.

Hammond (1999) in “Plant bioechnology; new products and applications” Eds. Hammond, McGarvey & Yusibov, Springer Verlag, NY 1999.

1. A process for producing a heterologous protein in a transgenic plant in a controlled environment, comprising:
cultivating hydroponically in a greenhouse transgenic plants in an inert soil-free matrix, which plants express in at least part of their tissue said heterologous protein,
conveying on a conveyor belt gutters containing the transgenic plants in said inert matrix, through different zones of irrigation with animal-free, chemically defined nutrition solution, and wherein newly sowed or planted plants are placed at the front end of said conveyor belt, and harvesting occurs at the back end of the conveyor belt,
harvesting said transgenic plants,
separating from said harvested plants said heterologous protein.

2. The process of claim 1, further comprising the steps of
sterilising transgenic seeds of said transgenic plant,
sowing said seeds in said inert soil-free matrix and allowing to germinate,
placing on the front end of said conveyor belt seedlings obtained after germination of said seeds.

3. The process of claim 1, wherein said inert soil-free matrix is selected from the group consisting of volcanic pumice, light expanded clay aggregate (LECA), rockwool, glasswool, perlite, coir, vermiculite, sterilized sand, washed gravel, and polystyrene peanuts.

4. The process of claim 1, wherein said plants express said heterologous protein in their seeds.

5. The process of claim 4, wherein said step of separating the heterologous protein from said harvested plants comprises threshing the plants and collecting the seeds of the plants.

6. The process of claim 1, wherein the conveyor belt passes through at least three zones.

7. The process of claim 6, wherein plants are conveyed through each of said zones for a period of about 2-6 weeks.

8. The process of claim 1, wherein nutrient concentration is varied between different zones.

9. The process of claim 1, wherein duration and/or frequency of irrigation is varied between zones.

10. The process of claim 1, wherein said isolation of the heterologous protein comprises collecting seeds from harvested transgenic plants, surface sterilising the seeds, and milling said seeds, to obtain a flour material comprising said heterologous protein.

11. The process of claim 1, wherein said transgenic plants are selected from the group consisting of dicotyledonous plants and monocotyledonous plants.

12. The process of claim 1, wherein said transgenic plants are selected from the group of plants consisting of tobacco, rape seed, soy bean, alfalfa, barley, maize, wheat, oat and rice.

13. The process of claim 1, wherein the said heterologous protein is selected from the group consisting of growth factors or cytokines.

14. The method of claim 1 where the heterologous protein is a growth factor selected from Transforming Growth Factors-b (or beta) (TGFs-b or TGFs-beta), Transforming Growth Factor-a (or alpha) (TGF-a or TGF alpha), Tumor Necrosis Factor alpha (TNF alpha), Epidermal Growth Factor (EGF), BMP-4, Platelet-Derived Growth Factor (PDGF), KGF, Fibroblast Growth Factors a and (aFGF and bFGF), Vascular Epithelial Growth Factor (VEGF) Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-7 (IL-7), Interleukin-6 (IL-6), Interleukin-8 (IL-8), Interleukin-10 (IL-10), Interleukin-18 (IL-18), Interleukin-20 (IL-20), Tumor Necrosis Factor-a (TNF-a), Tumor Necrosis Factor-b (TNF-b), Interferon-g (INF-g),Granulocyte Colony Stimulating Factor (G-CSFs) ,Granulocyte Macrophage Colony Stimulating Factor (GM-CSF), Macrophage Colony stimulating factor (M-CSF), Nerve Growth Factor (NGF), Keratinocyte Growth Factor (KGF), Bone morphogenesis Protein (BMP-4), and Thymosin beta 4.

15. A system for producing heterologous protein from transgenic plants in a greenhouse, comprising:
transgenic plants expressing in at least a portion of their tissue said heterologous protein,
a conveyor belt with gutters for holding said plants in soil-free inert matrix, selected from the group of volcanic pumice, light expanded clay aggregate (LECA), rockwool, glasswool, perlite, coir, vermiculite, sterilized sand, washed gravel, and polystyrene peanuts,
an irrigation system for delivering water and/or a animal-free nutrient solution to said plants, said irrigation system divided into zones such that for each zone a different composition and/or concentration of nutrients and/or a different irrigation scheme, can be implemented.

16. The system of claim 15, wherein said plants express in their seeds said heterologous protein.
